(19) United States
(12) Patent Application Publication
(10) Pub. No.: US 2010/0222294 A1
(43) Pub. Date: Sep. 2, 2010

(54) Formulations of ATP and Analogs of ATP

(75) Inventor: Amir Pelleg, Haverford, PA (US)

Correspondence Address: FISH & RICHARDSON P.C., P.O. BOX 1022, MINNEAPOLIS, MN, 55440-1022, US

(73) Assignee: DUSKA SCIENTIFIC CO., Philadelphia, PA (US)

(21) Appl. No.: 12/715,170

(22) Filed: Mar. 1, 2010

Related U.S. Application Data
(60) Provisional application No. 61/156,263, filed on Feb. 27, 2009.

(51) Int. Cl. A61K 31/7076; A61P 35/00; A61P 9/12; A61P 9/10; A61P 9/00; A61P 11/06; A61P 11/08; C12N 5/02; A01N 1/02

(52) U.S. Cl. 514/47; 435/375; 435/2

(57) ABSTRACT

This disclosure provides solutions and compositions (e.g., pharmaceutical solutions and compositions) containing adenosine 5′-triphosphate (ATP) or an analog thereof. In addition, it features methods of making and using the solutions and compositions.

This application claims priority of U.S. Provisional Application No. 61/156,263, filed Feb. 27, 2009, the entire disclosure of which is incorporated herein by reference in its entirety.

### TECHNICAL FIELD

This invention relates to compositions containing adenosine 5′-triphosphate (ATP), or analogs thereof, and more particularly to pharmaceutical compositions containing ATP or analogs thereof.

### BACKGROUND

Many therapeutic, prophylactic, and diagnostic uses for ATP, or analogs thereof, have been developed. There is a need for stable liquid pharmaceutical formulations of ATP and analogs thereof in which their pharmacological activity is maintained.

### SUMMARY

This disclosure is based on the findings of the inventors that an alkaline solution of ATP is stable for at least six years. The disclosure features stable solutions and pharmaceutical compositions containing ATP, or an analog thereof, and methods of making and using such solutions and pharmaceutical compositions.

More specifically, the disclosure provides a first aqueous solution containing: an aqueous solvent; an adenosine 5-triphosphate (ATP) reagent; and glycine, the solution having a pH of between about 8.7 and about 9.5.

Another aspect of the disclosure is a second aqueous solution that contains: an aqueous solvent; and an ATP reagent, the solution having a pH of about 8.7 to about 9.5 and being formulated for administration to a subject or for contacting a mammalian cell with the ATP reagent.

Also featured by the disclosure is a third aqueous solution containing: an aqueous solvent; and ATP, the solution having a pH of between about 8.7 and about 9.5. The solution is such that, at the end of a period of time at about 4° C. to about 8° C., it contains an amount of ADP that is not more than about 5.0% (e.g., not more than about 4.0%, not more than about 3.0%, or not more than about 2.5%) of the amount of ATP in the solution, the period of time being up to six years after the solution was made.

In the solutions of the disclosure the ATP reagent can be ATP or a pharmaceutically acceptable salt thereof. It can also be an ATP analog or a pharmaceutically acceptable salt thereof. The second and third aqueous solutions can further contain glycine. The pH of any of solutions of the disclosure can be between about 8.7 and about 9.4, e.g., about 8.8 and about 9.3. Moreover, any of the solutions can further contain a biocompatible buffer, e.g., a phosphate buffer such as a phosphate buffer that contains Na₂HPO₄ and/or K₂HPO₄. The biocompatible buffer can also be a bicarbonate buffer, an acetate buffer, a citrate buffer, or a glutamate buffer. In addition, any of the solutions can contain one or more of 1,3-Bis[tris(hydroxymethyl)methylamino]propane (Bis-Tris Propane); Tris(hydroxy)aminomethane (Tris); Tris(hydroxymethyl)aminomethane (Trizma); 4-(2-Hydroxyethyl)-1-piperazinepropanesulfonic acid (EPPS); N-[Tris(hydroxymethyl)methyl]glycine (Tricine); glycine; Diglycine (Gly-Gly); N,N-Bis(2-hydroxyethyl)glycine (Bicine); N-(2-Hydroxyethyl)piperazine-N′-(4-butanesulfonic acid) (HEPBS); N-[Tris(hydroxymethyl)methyl]-3-aminopropanesulfonic acid (TAPS); 2-Amino-2-methyl-1,3-propanediol (AMPD); N-tris(Hydroxymethyl)methyl-4-aminobutanesulfonic acid (TABS); N-(1,1-Dimethyl-2-hydroxyethyl)-3-amino-2-hydroxypropanesulfonic acid (AMPSO); 2-(Cyclohexylamino)ethanesulfonic acid (CHES); 3-(Cyclohexylamino)-2-hydroxy-1-propanesulfonic acid (CAPSO); or β-Aminoisobutyl alcohol (AMP).

Any of the solutions can further contain a stabilizer. The stabilizer can be a chelating agent, e.g., ethylenediaminetetraacetic acid (EDTA) or ethylene glycol tetraacetic acid (EGTA). The stabilizer can also be a sugar alcohol (e.g., sorbitol, mannitol, adonitol, erythritol, xylitol, lactitol, isomalt, maltitol, or a cyclitol), glycerol, methionine, or creatinine.

The ATP analog in any of the solutions can be: α,β-methylene-ATP (α,βmATP); β,γ-methylene-ATP (β,γmATP); 2-thio-ATP (2-SH-ATP); 2-methylthio-ATP (2-MeS-ATP); 2′,3′-O-2,4,6,-trinitrophenyl-ATP (TNP-ATP); 2′,3′-O-(4-benzoyl)-ATP (BzATP); an N-alkyl-2 ATP; adenosine 5′-(β,γ-imido)triphosphate (AMP-PNP); ATP-MgCl₂; or oxidized ATP (oATP). The concentration of ATP, or the analog thereof, in the solutions can be about 18.15 mM or about 36.30 mM. The concentration of glycine can be about 13.32 mM. The solution can be one that includes: 18.15 mM ATP; and Na₂HPO₄. Any of the solutions can be formulated for parenteral administration to a subject, e.g., for administration to a subject by injection. Alternatively, the solutions can be formulated for intravenous administration, for enteral (e.g, oral) administration, for topical administration, or for transdermal administration to a subject.

In another embodiment, the disclosure provides a method of making an aqueous solution. The method includes: mixing together water, glycine, and an ATP reagent to create a mixture; and adjusting the pH of the mixture to between about 8.7 to about 9.5 to create the solution. The method can further involve mixing into the mixture a biocompatible buffer and/or mixing into the mixture a stabilizer. The pH can be adjusted by adding a base, e.g., sodium hydroxide, to the mixture. The method can also further include storing the solution at a temperature of between about 4° C. and about 8° C. for a period of time of up to six years. Where the ATP reagent is ATP, at the end of the period of time, the solution can contain an amount of ADP that is not more than about 5.0% of the amount of ATP in the solution.

Another aspect of the disclosure is an in vitro method of delivering an ATP reagent to a mammalian cell. The method includes incubating the cell with a medium that contains any of the above solutions in vitro. The mammalian cell can be, for example, a spermatozoon.

Also provided by the invention is an in vivo method of contacting a mammalian cell in a mammalian subject with an ATP reagent. The method includes administering a composition containing or being any of the solutions disclosed herein to the subject, e.g., a human subject. The method can be a therapeutic method, a prophylactic method, or a diagnostic method. In the method, the subject can have, be suspected of having, or be at risk of developing a condition selected from an obstructive pulmonary disease (OPD), asthenozoospermia, pain, tissue injury, nerve damage, organ failure, a condition requiring reduction in blood pressure, pulmonary hypertension, tachycardia, myocardial ischemia, coronary artery disease, cystic fibrosis, cancer, and cancer-related cachexia. The cell can be a neuron (e.g., a retinal neuron, a cortical neuron, a hippocampal neuron, a basal ganglion neuron, a spinal cord neuron, a pulmonary vagal C fiber, or a pulmonary vagal A fiber), a spermatozoon, a vascular smooth muscle cell, or a vascular endothelial cell. Moreover the cell can be a cancer cell or a normal cell. The OPD can be chronic obstructive pulmonary disease (COPD), chronic asthma, acute bronchitis, emphysema, chronic bronchitis, bronchiectasis, cystic fibrosis, cough, or acute asthma. The method can be a method to determine whether the subject has COPD or asthma or a method for assessing the efficacy of a treatment for an OPD. Where the cell is a spermatozoon, the method can further involve testing the motility of the spermatozoon. The testing can be, e.g., before or after the contacting.

Another aspect of the disclosure is a kit containing any of the solution described herein and instructions for administering the solution to a subject.

Another kit can contain an ATP reagent, glycine, and instructions for making any of the solutions described in the disclosure. The kit can further contain Na₂HPO₄.

As used herein, “biocompatible” means compatible with a living tissue or living system by not being toxic, injurious, or inflammatory, and/or not causing an immunological response.

The phrase “pharmaceutically acceptable” is employed herein to refer to those compounds, materials, compositions, solutions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “adenosine 5′-triphosphate” or “ATP” refers to ATP or a hydrate thereof. The term “ATP analog” refers to any ATP analog listed below or a hydrate thereof. In addition, the term “ATP reagent” refers to ATP, any of the ATP analogs listed below, any pharmaceutically acceptable salt of ATP, and any pharmaceutically acceptable salt of any of the ATP analogs listed below.

A subject “suspected of having a condition” is one having one or more symptoms of the condition. Symptoms of conditions that are susceptible to treatment and/or prophylaxis by the in vivo methods of contacting a cell with an ATP reagent (see below), are familiar to those in the art. As used herein, a subject “at risk of developing a condition” is a subject that has a predisposition to develop the condition (i.e., a genetic predisposition to develop the condition (e.g., a family history of the condition)) or has been exposed to an environment (e.g., a high level of ionizing radiation or excessive ultra-violet light) or substances (e.g., chemical carcinogens, tobacco smoke, or a long-term high-fat diet) that can result in the condition. Known appropriate predispositions and environments are familiar to those in the art. As is clear from the above definitions, subjects of a species of interest that are suspected of having, or are at risk of developing, a particular condition are not all members of that species.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., stable ATP formulations, will be apparent from the following description, from the drawings and from the claims.

### DESCRIPTION OF DRAWINGS

FIG. 1 is a depiction of the chemical structure of ATP. Greek lettering (α, β, and γ) shows the phosphorus atoms of the relevant phosphate groups at the 5′ position.

### DETAILED DESCRIPTION

The inventor has found that an alkaline solution of ATP, after storage for 6 years in a refrigerator maintained at a temperature of about 4° C. to about 8° C., contained adenosine 5′-diphosphate (ADP) at a concentration that was only 2.9% of the concentration of ATP in the solution at that time. Below are described various stable solutions of ATP reagents that can be stored for extended periods of time without a substantial decrease in the relative amount of ATP reagents in the solution. Also described below are compositions of ATP reagents that are formulated for administration to a mammalian subject (e.g., a human subject) or for contacting with a mammalian cell with the ATP reagent. In addition, the disclosure provides methods of making and using the solutions and compositions.

The main form of degradation of ATP is hydrolysis from ATP to adenosine 5′-diphosphate (ADP) and, to some degree, from ADP to adenosine 5′-monophosphate (AMP) and from AMP to adenosine.

This disclosure provides aqueous solutions containing one or more (e.g., two, three, four, five, six, seven, eight, nine, ten, 15, or 20) ATP reagents. The solutions have a pH of about 8.7 to about 9.5 (e.g., about 8.7 to about 9.4, about 8.8 to about 9.3). Thus, the pH of the solutions can be about 8.7, about 8.8, about 8.9, about 9.0, about 9.1, about 9.2, about 9.3, about 9.4, about 9.5 or any range of pH's in which the lower and upper limits are selected from these values. As used in the context of these pH values, the term “about” means within 1.6% to 1.8% (i.e., 1.6%, 1.65%, 1.7%, 1.75%, or 1.8%) of the stated value. The pH of the solutions can be 8.7, 8.8, 8.9, 9.0, 9.1, 9.2, 9.3, 9.4, or 9.5, or any range of pH's in which the lower and upper limits are selected from these values.

All of the aqueous solutions of this disclosure can contain any aqueous solvent. In certain instances, the solvent is biocompatible and/or pharmaceutically acceptable. The aqueous solvent can be water alone or water with any one or more of a wide variety of additives that are either biocompatible and/or pharmaceutically acceptable and/or are such that the resulting solution can be contacted with a vertebrate cell without damaging (e.g., lysing) the cell. In the latter case the solution can be isotonic, i.e., the solution will have the same total concentration of solutes as exists inside relevant vertebrate cells with which the solution will be contacted. The mammalian cell is any kind of mammalian cell (e.g., a neuron or a spermatozoon) and can be a normal, non-malignant cell or a cancer (malignant) cell (see below). The cell can be of any of the species listed herein.

Any of the solutions can have the property of containing, at the end of a period of time at about −5° C. to about 10° C.; about 0° C. to about 10° C.; about 2° C. to about 10° C.; about 4° C. to about 10° C.; about 0° C. to about 8° C.; about 2° C. to about 8° C.; or about 4° C. to about 8° C., an amount of ADP, or a corresponding degradation product of an ATP analog, that is not more than 5% (e.g., not more than: 4.5%; 4%; 3.5%; 3%; 2.5%; 2%; 1.5%; or 1%) of the amount of ATP (or the ATP analog) in the solution, the period of time being any period of time up to six years (e.g., one day, two days, three days, a week, two weeks, a month, two months, three months, six months, nine months, a year, 18 months, two years, three years, four years, five years, six years, or any range of time in which the upper and lower limits are selected from these values) after the solution was made. As used in the context of the above temperatures, the term “about” means within 1% to 5% (i.e., 1%, 2%, 3%, 4%, or 5%) of the stated value. The temperature at which the solutions can be stored for the period of time can be 0° C., 1° C., 2° C., 3° C., 4° C., 5° C., 6° C., 7° C., 8° C., 9° C., or 10° C., or any range of temperatures in which the lower and upper limits are selected from these values. It is understood that the solutions of the disclosure can be stored for any period of time desired, e.g., a time exceeding six years, and at any temperature desired. All that is required is in the above-stated embodiment is that, if the solution is stored for up to six years at temperature within the above stated range, it will contain only the above-stated relative amount of ADP, or a corresponding degradation product of an ATP analog.

As used herein, the time when a “solution was made” is the day a relevant ATP reagent was added to the aqueous solvent containing all or some of the other components of the solution (see below for methods of making solutions and compositions containing ATP reagents).

Any of the solutions can be formulated for administration to a mammalian subject (see below) or for contacting a mammalian cell with the ATP reagent.

In certain instances, the solutions described herein include one or more additives. Examples of additives useful in the solutions described herein include buffers or buffering agents (e.g., glycine), salts, and stabilizers. In certain instances, the additive is a biocompatible and/or pharmaceutically acceptable.

The concentration of one or more of the additives (e.g., glycine) in the solutions of this disclosure can be any convenient concentration. It can be, for example, from about 0.1 mM to about 1.0 M, e.g., about 1 mM, about 10 mM, about 25 mM, about 50 mM, about 75 mM, about 100 mM, about 200 mM, or about 500 mM, or any range of concentrations in which the lower and upper limits are selected from these values. As used in the context of the above concentrations, the term “about” means within 1% to 10% (i.e., 1%, 2%, 3%, 4%, 5%, 6%, 7%, 8%, 9%, or 10%) of the stated value. In the examples below glycine is present in solution at a concentration of 13.32 mM.

Numerous buffers and buffering agents are well known in the art. The selection of the proper buffering agent is within the skill of an ordinary artisan. Suitable buffers include any buffer that can be used to provide a solution having a pH of about 8.7 to about 9.5. Examples include, without limitation, phosphate buffers (containing, for example, NaH₂PO₄ and Na₂HPO₄; or KH₂PO₄ and K₂HPO₄; Na₂HPO₄ and citric acid), borax buffers (containing, for example, borax and NaOH). Other common buffering agents include, without limitation, 1,3-Bis[tris(hydroxymethyl)methylamino]propane (Bis-Tris Propane); Tris(hydroxy)aminomethane (Tris); Tris(hydroxymethyl)aminomethane (Trizma); 4-(2-Hydroxyethyl)-1-piperazinepropanesulfonic acid (EPPS); N-[Tris(hydroxymethyl)methyl]glycine (Tricine); glycine; Diglycine (Gly-Gly); N,N-Bis(2-hydroxyethyl)glycine (Bicine); N-(2-Hydroxyethyl)piperazine-N′-(4-butanesulfonic acid) (HEPBS); N-[Tris(hydroxymethyl)methyl]-3-aminopropanesulfonic acid (TAPS); 2-Amino-2-methyl-1,3-propanediol (AMPD); N-tris(Hydroxymethyl)methyl-4-aminobutanesulfonic acid (TABS); N-(1,1-Dimethyl-2-hydroxyethyl)-3-amino-2-hydroxypropanesulfonic acid (AMPSO); 2-(Cyclohexylamino)ethanesulfonic acid (CHES); 3-(Cyclohexylamino)-2-hydroxy-1-propanesulfonic acid (CAPSO); or β-Aminoisobutyl alcohol (AMP). In some embodiments, the buffering agent can include one or more of glycine or Na₂HPO₄.

Any of a wide range of biocompatible and/or pharmaceutically acceptable salts known to those in the art can be included in the solutions. These include, without limitation, sodium (Na), potassium (K), lithium (Li), calcium (Ca), magnesium (Mg), and manganese (Mn) salts. The salts can be, without limitation, chloride, bromide, fluoride, sulphate, sulfite, carbonate, bicarbonate, borate, phosphate, or vanadate salts. Examples of salts of interest include, without limitation, NaCl, KCl, LiCl, MgCl₂, Na₂SO₄, K₂SO₄, CaSO₄, and the phosphate salts listed above under buffers.

As used herein, “stabilizers” are substances that prevent decomposition, or decrease the rate of decomposition, of ATP reagents. Suitable stabilizers can function by any of a variety of mechanisms and the instant invention is not limited by any particular mechanism by which they can act. In certain instances, the stabilizer is biocompatible and/or pharmaceutically acceptable. Exemplary stabilizers include, but are not limited to ethylenediaminetetraacetic acid (EDTA) and ethylene glycol tetraacetic acid (EGTA), ethylenediaminedisuccinic acid (EDDS), diethylene triamine pentaacetic acid (DTPA), hydroxyethylethylenediaminetriacetic acid (HEDTA), nitrilotriacetic acid (NTA), dimercaprol, dimercapto-propane sulfonate, dimercaptosuccinic acid, glycine, malic acid, oxalic acid, citric acid, ascorbic acid, diethylene triamine penta (methylene phosphinic acid (DTPMP), alpha lipoic acid, aminophenoxyethane-tetraacetic acid (BAPTA), deferasirox, deferiprone, deferoxamine, (or salts thereof), and polyhydroxyl compounds (also sometimes referred to as polyhydroxy or polyol compounds).

Useful stabilizers can also be carbohydrates (e.g., monosaccharides, disaccharides, oligosaccharides, and polysaccharides), sugar alcohols, glycerol, poly-glycerol, ethylene glycol, propylene glycol. polyethylene glycol (PEG), and polyvinyl alcohols. Esters of these polyhydroxyl compounds are also useful as stabilizers. Suitable sugar alcohols include, without limitation, sorbitol, mannitol, adonitol, erythritol, xylitol, lactitol, isomalt, maltitol, or a cyclitol. Other polyhydroxyl compounds (and ester derivatives thereof) useful as stabilizers are those listed in U.S. Pat. No. 5,284,655, the disclosure of which is incorporated herein by reference in its entirety. Additional useful stabilizers include, starch derivatives (e.g., maltodextrins, hydroxyethyl starch (HES), or hydrogenated starch hydrolysates (HSH)), hyaluronic acid, chondroitin sulfate, methionine, and creatinine.

It will be appreciated that the above categories of solution components are not mutually exclusive. Thus, for example, a given buffering component can also be a salt and/or a stabilizer.

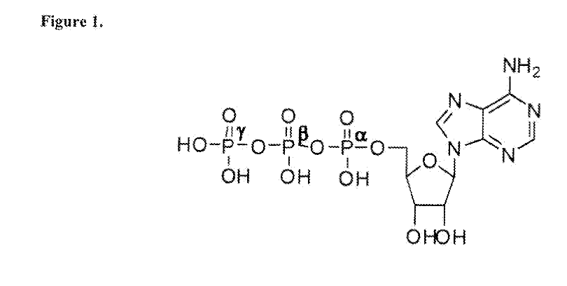

The chemical structure of ATP is shown in FIG. 1. As used herein, the term “analogs of ATP” or “ATP analogs” refers to all of the ATP-related compounds listed below. The term “analogs of ATP” or “ATP analogs” can refer to ATP agonists and/or ATP antagonists. They can bind reversibly or irreversibly to cellular receptors for ATP (P2 purinergic receptors). The category of P2 receptor depends on the cell involved. P2R are divided into two families: P2X, ligand-binding, dimeric, trans-cell membrane cationic channels, and P2Y, seven trans-cell membrane domain G protein-coupled receptors. Eight P2Y (P2Y₁, P2Y₂, P2Y₄, P2Y₆, P2Y₁₁, P2Y₁₂, P2Y₁₃, and P2Y₁₄), seven homodimeric P2X receptor subtypes (P2X₁₋₇), and five P2X heterodimeric receptors (PX_{1/2}, PX_{2/3}, PX_{2/6}, PX_{1/5}, and PX_{1/6}) have been identified and cloned. In general, the stimulation of the P2Y receptors activates an intracellular signal transduction pathway culminating in the increase in the level of intracellular calcium (Ca²⁺) ions.

Analogs of ATP useful in the solutions of the disclosure can include ATP molecules with a modified phosphate side chain, ribose moiety, and/or base (adenine) moiety (see, e.g., Bagshaw, C. R. (2001) J. Cell. Sci., 114(3): 459, which is incorporated by reference in its entirety herein).

Examples of analogs of ATP with modified phosphate side chains include compounds:
wherein the O atom between the β and the γ P atoms of ATP is replaced with —NH to produce adenosine 5′-(β,γ-imido)triphosphate) (AMP-PNP; see e.g., Table 1, 1), or with a methylene group to produce β,γ-methylene-ATP (β,γmATP; see e.g., Table 1, 2);
wherein the O atom between the α and βP atoms of ATP is replaced with a methylene group to produce α,β-methylene-ATP (α,βmATP; see e.g., Table 1, 18);
wherein the O atoms are complexed with Mg²⁺ ions, such as in the form of MgCl₂ (ATP-Mg);
wherein the γ phosphate moiety of ATP is replaced with a vanadate moiety to produce γ-vanadate analogs (ADP.Vt; see e.g., Table 1, 15); and
wherein the oxygen double bonded to the γP of ATP is replaced with a double bonded sulfur atom to produce thio-ATP analogs (ATPγS; see e.g., Table 1, 7).

Additional examples of ATP analogs where the phosphate side chain is modified are shown in Table 1.

ATP analogs can also be hydrolysable or non-hydrolysable under physiological conditions. ATP is generally hydrolyzed between the β and γ phosphate groups, and modification of the phosphate side chain can produce analogues which are hydrolysable, slowly hydrolysable, or non-hydrolysable compared to ATP. For example, under certain conditions, thio-ATP analogs are more slowly hydrolyzed than ATP; α,β-methylene-ATP has a modified phosphate chain, but is hydrolysable; and ATP-Mg is hydrolysable and has been used in a variety of clinical applications (see, e.g., Agteresch et al. (1999) Drugs 58(2): 211-232, the disclosure of which is incorporated herein by reference in its entirety).

ATP analogs can also include ATP molecules where the ribose moiety has been modified (e.g., at one or both of the 2′ and 3′ positions). Exemplary analogs include 2′,3′-O-2,4,6,-trinitrophenyl-ATP (TNP-ATP) and 2′,3′-O-(4-benzoyl)-ATP (BzATP). The former compound is non-hydrolysable compared to ATP under physiological conditions.

Additional ribose modified ATP analogs include, but are not limited to 2′,3′-β-(cyanine Cy3)(ethylenediamine)-ATP (Cy3-EDA-ATP), 2′,3′-O-(cyanine Cy5)(ethylenediamine)-ATP (Cy5-EDA-ATP), 2′,3′-O-(fluorescein)(ethylenediamine)-ATP (FEDA-ATP), 2′,3′-O-(caged fluorescein)(ethylenediamine)-ATP (caged FEDA-ATP), 2′,3′-O-(rhodamine)(ethylenediamine)-ATP (REDA-ATP), 2′,3′-O-(dansyl)(ethylenediamine)-ATP (DEDA-ATP), 2′,3′-O-(diethylcoumarin)(ethylenediamine)-ATP (Cou-EDA-ATP), 2′,3′-O-(alexa532)(ethylenediamine)-ATP (alexa⁵³²-EDA-ATP), 2′,3′-O-(bodipy μL)(ethylenediamine)-ATP (bodipy FL-EDA-ATP), and 2′,3′-O-(bodipy TR)(ethylenediamine)-ATP (bodipy TR-EDA-ATP). ATP analogs are described in greater detail in Bagshaw (2001) J. Cell Sci. 114 (Pt. 3):459-460 and the poster insert (both the article and the poster are incorporated herein by reference in their entirety).

ATP analogs can also include compounds according to Formula I:

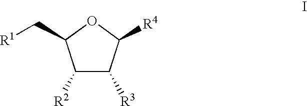

wherein:
R¹ is selected from the side chains shown in Table 1;

### R² is OR⁵;

### R³ is OR⁵ or NR⁵;

R⁴ is adenine or

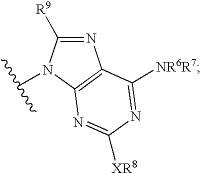

each R⁵ is independently H or a label;
X is S, N(R¹⁰) or a bond;
each R⁶, R⁷, and R⁸ is independently hydrogen, halogen, alkyl, aryl, aralkyl, —C(O)R¹¹, —C(O)N(R¹¹)(R¹¹), —C(O)OR¹¹—C(NR¹¹)N(R¹¹)(R¹¹), —S(O)₂R¹¹, —S(O)₂N(R¹¹)(R¹¹) or a label;
R⁹ is hydrogen, halogen, azide, or a label;
R¹⁰ is hydrogen, alkyl, aryl, or aralkyl; and
each R¹¹ is independently hydrogen, alkyl, aryl, or aralkyl; or two R¹¹ come together to form a 4 to 7 membered heterocyclic ring;
or a salt thereof;
wherein at least one of R¹, R², R³ or R⁴ is not as found in ATP.

TABLE 1 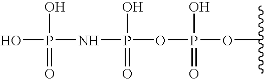
1 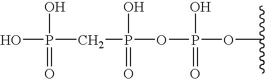
2 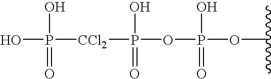
3 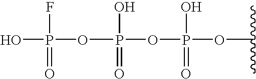
4 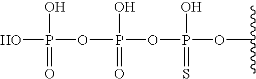
5 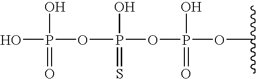
6 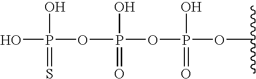
7 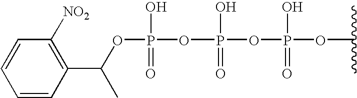
8 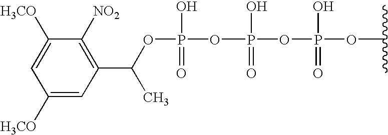
9 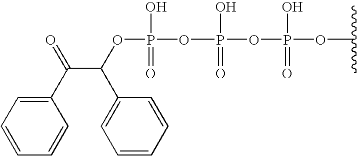
10 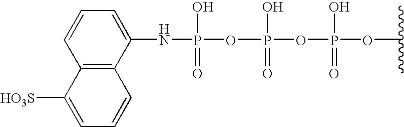
11 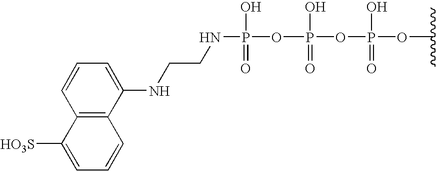
12 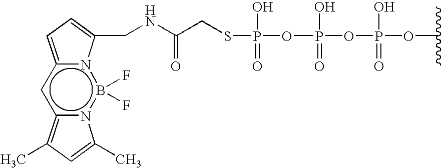
13 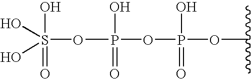
14 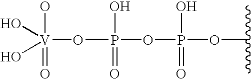
15 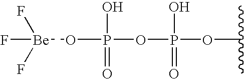
16 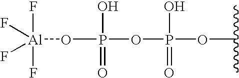
17 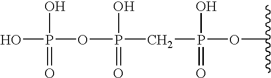
18 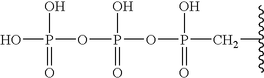
19 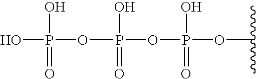
20 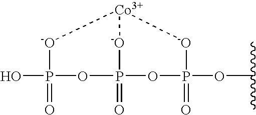
21 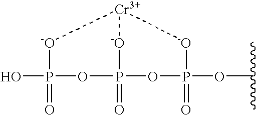
22

Labels, as described herein, can include any moiety that can be used as a fluorescent label, a luminescent label, an NMR probe, an EPR probe, a spectroscopic probe, LRET probe, a photoaffinity label, in Electron microscopy, or is photoactivatable. Non-limiting examples include formycin; 2-aminopurine; aminonaphthyl-suphonyl; dansyl; anthraniloyl; methyl-anthraniloyl; diethylcoumarin; trinitrophenyl; fluoroescein; caged fluorescein; biotin; bodipy FL; bodipy TR; alexa532; rhodamine; cyanine Cy3; cyanine Cy5; TEMPO; and dibenzyl. The label can be directly linked to the ATP moiety or linked through a linker. A linker moiety is any physiologically compatible chemical group that does not interfere with the functions of the label or ATP. Preferred linkers are synthetically easy to incorporate into the contrast agent. They are also not so unduly large as to manifest their own undesired biological function or targeting influence onto the label or ATP. Preferably, the length of the linker is between 1 and 50 angstroms, more preferably 1 and 10 angstroms. Non-limiting examples of linkers include C₁₋₂₀ alkyl; C₁₋₂₀ alkene; ethylenediamine (EDA); diethylenetriamine (DETA); triethylenetetramine (TETA); tetraethylenepentamine (TEPA); pentaethylenehexamine (PEHA); diaminohexane; tetramethylethylenediamine (TMEDA); and tetraethylethylenediamine (TEEDA).

In summary, the ATP analogs of the present disclosure can include ATP molecules, where one or more of the phosphate chain, ribose moiety, or base (adenine) has been modified. In certain instances, the ATP analog is selected from the group consisting of oATP; AMP-PNP; β,γmATP; ADP.Vt; ATPγS; α,βmATP; ATP-Mg; ATP-MgCl₂; TNP-ATP; BzATP; 2-SH-ATP; and 2-MeS-ATP.

The ATP reagents useful in the compositions described herein may contain a basic functional group, such as an amino group, and are thus capable of forming salts with acids. These salts can be prepared in situ in the ATP solutions described herein or the ATP solution manufacturing process, or by separately treating the ATP reagent in its free base form with a suitable organic or inorganic acid, and isolating the salt thus formed during subsequent purification. Representative salts include salts derived from suitable inorganic and organic acids, e.g., hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other suitable acid addition salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like (see, for example, Berge et al. (1977) “Pharmaceutical Salts”, J. Pharm. Sci. 66:1-19).

In certain cases, the ATP reagents useful in the compositions described herein may contain one or more acidic functional groups and, thus, are capable of forming salts with bases. These salts can be prepared in situ in the ATP solutions described herein or the ATP solution manufacturing process, or by separately treating the ATP reagent in its free acid form with a suitable base, such as the hydroxide, carbonate or bicarbonate of a alkali or alkaline earth cation, with ammonia, or with a organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

In certain instances, the ATP reagents described herein further comprise metals coordinated to the ATP reagent. For example, a metal can coordinate to one or more of the Lewis bases present in the ATP reagent (e.g., an oxygen present in the phosphate side chain of the ATP reagent). The metal can be any metal, including but not limited to alkali metals, alkaline earth metals (e.g., Mg²⁺ or Ca²⁺), lanthanides, actinides, and transition metals (e.g., Cr³⁺ or Co³⁺). Exemplary coordinated metal complexes include Co³⁺-ATP, Cr³⁺-ATP, and MgCl₂-ATP.

The concentration of ATP reagents in the solutions of this disclosure can be any convenient concentration. It can be, for example, from about 0.1 mM to about 1.0 M, e.g., about 1 mM, about 10 mM, about 25 mM, about 50 mM, about 75 mM, about 100 mM, about 200 mM, or about 500 mM, or any range of concentrations in which the lower and upper limits are selected from these values. As used in the context of the above concentrations, the term “about” means within 1% to 10% (i.e., 1%, 2%, 3%, 4%, 5%, 6%, 7%, 8%, 9%, or 10%) of the stated value. In certain instances, the concentration of the ATP reagent is 18.15 mM or 36.30 mM.

The disclosure also provides methods of making solutions and compositions containing ATP reagents. In these methods, one or more ATP reagents are added to an aqueous solvent (water or water containing all or some of the components of the solution or composition) that is a component of the solution or composition. Components that can be in the solutions and compositions of this disclosure are listed above and below. Where the ATP reagent(s) is/are added to water or water containing only some of the components of the solution or composition, other components can be added after the addition of the ATP reagent(s). Where the ATP reagent(s) is/are added in solid form, it dissolves in the aqueous solvent or the solution or composition after addition of all the components. It is understood, that the ATP reagents can also be added to the aqueous solvent of the solution in the form of a previously made solution or suspension (e.g., a stock solution or stock suspension) that can have been stored frozen.

With respect to solutions, the pH can be adjusted to a desired pH (see above) using an appropriate basic solution (e.g., a solution of NaOH, KOH, LiOH, Ca(OH)₂, or Mg(OH)₂ in water) or an acid solution (e.g., HCl, H₂SO₄, or acetic acid diluted in water). The pH adjustment can be performed after addition of all the components but can be performed before the addition of all of the water and one or more other components.

The solutions and compositions of this disclosure can be sterilized (e.g., for use as therapeutic compositions or for in vitro applications) by any of a variety of methods. Sterile solutions and compositions are not limited by any particular method of sterilization. Thus, sterilization can be achieved by, for example, exposure to sterilizing (but not ATP reagent-degrading) doses of radiation (e.g., x-radiation, γ-radiation, e-beam radiation, or ultraviolet (UV) light) or by microfiltration.

It will be appreciated that microfiltration is generally only applicable to solutions and compositions in which all components are dissolved in an appropriate solvent. It can be performed after all components have been added to an appropriate solution or composition. In addition, water used to make the solutions or compositions can be microfiltered before any components have been added and the components can have been sterilized by another method, e.g., irradiation (see above). Moreover, more than one (e.g., two, three, four, or five) methods of sterilization can be used to obtain a sterile solution or composition and these can be applied to any and all of the components and at any stage of making the solution or composition.

It is understood that while the solutions of this disclosure have a pH of about 8.7 to about 9.5 (see above), compositions (e.g., pharmaceutical compositions useful in the methods described below) made by mixing a solution of the disclosure with one or more additional additives (see below) can have any desired pH (e.g., a neutral pH). The pH of a composition can result from adding the relevant additives and/or by adjusting the pH as described for the solutions in the previous paragraph. The pH of a solution can be adjusted to create a composition having an appropriate pH as part of a manufacturing process or just before administration to a subject by, e.g., a medical service provider such as a nurse or a doctor.

Methods of Contacting a Cell with an ATP Reagent

The disclosure features a variety of in vitro, in vivo, and ex vivo methods of contacting a cell with an ATP reagent.

In the methods of this disclosure, the cell can be any mammalian cell that has a cell surface receptor for an ATP reagent or that is responsive to an ATP reagent by some mechanism not involving a cell surface receptor. Cell-surface receptors targeted by the methods of this disclosure include any of the P2 purinergic receptors disclosed herein. Such cells can be benign (non-malignant) cells or cancer (malignant) cells. Non-malignant cells include, but are not limited to, neurons (central nervous system (CNS) or peripheral nervous system (PNS) neurons), smooth muscle cells (e.g., vascular smooth muscle cells), endothelial cells (e.g., vascular endothelial cells), or spermatozoa. Neurons include retinal neurons (e.g., retinal ganglion cells), cortical neurons, hippocampal neurons, basal ganglia neurons, spinal cord neurons, or pulmonary vagal sensory nerve fibers (e.g., C or A fibers). Cancer cells include cancer cells that have the above-recited properties. They can be, without limitation, cells of hematological cancers (e.g., T and B cell lymphomas, leukemias, mastocytomas), dermatological cancers (e.g., basal cell carcinomas, squamous cell cancers, or melanomas), gastrointestinal cancers (e.g., esophageal, stomach, colon, or rectal cancers), breast cancers, pancreatic cancer cells, lung cancer cells (e.g., small cell lung cancers and nonsmall cell lung cancers), sarcomas (e.g., fibrosarcomas), genitourinary cancer cells (e.g., renal cancers, bladders cancers, ureter cancers, prostate cancers, penile cancers, testicular cancers, ovarian cancers, cervical cancers, uterine cancers, vaginal cancers, and urethral cancers), liver cancers, head and neck cancers, and neurological cancers (e.g., malignant gliomas and astrocytomas). The cells can be from or in a subject of any of the species listed below.

Where it is desired to generate an ATP response in a cell, the cell is contacted with ATP or an agonistic ATP analog. On the other hand, where it is desired to prevent or inhibit an ATP mediated response in a cell, the cell is contacted with an antagonistic ATP analog.

### In Vitro Methods

In vitro methods involve incubating a cell of interest with any of the ATP reagent solutions described above or in a medium (e.g., an isotonic medium such as normal saline (NS) or phosphate buffered saline (PBS) or tissue culture medium) to which one of the ATP reagent solutions has been added. In instances where the cell is incubated in the ATP reagent solution, the solution can be isotonic. The in vitro methods can be components of basic scientific studies on, for example, an ATP reagent and its effects on cells. Moreover, the ATP reagent solutions can be used to test for cytotoxic activity against cells of interest, e.g., any of the cancer cells listed above. In addition, such in vitro methods can be “positive controls” in screening assays to test for an activity known to be mediated by ATP (or an ATP analog) in other compounds of interest.

Furthermore, spermatozoa (e.g., spermatozoa from an asthenozoospermic subject) can be cultured in a solution or composition containing ATP, or an agonistic ATP analog, in order to increase the motility and hence fertilizing potential of the spermatozoa. The spermatozoa can then be used in artificial insemination and/or in vitro fertilization methods. Spermatozoa so contacted can be tested for relative motility before and/or after the contacting. For further details of such methods, see published International Patent Application Publication No. WO 2008/106527, the disclosure of which is incorporated herein by reference in its entirety.

### In Vivo Methods

In the in vivo methods of contacting a cell with an ATP reagent, one of the above-described solutions, or a composition containing such a solution, is administered to a mammalian subject. The mammalian subject can be, for example, a human (e.g., a human patient) or non-human primate (e.g., chimpanzee, baboon, or monkey), mouse, rat, rabbit, guinea pig, gerbil, hamster, horse, a type of livestock (e.g., cow, pig, sheep, or goat), dog, or a cat. The subject can be one having, suspected of having, or at risk of developing a relevant pathological condition (see below).

The methods can be diagnostic, therapeutic, or prophylactic. Prophylaxis can be achieved by administering appropriate solutions or compositions to subjects suspected of having or likely to develop a relevant pathological condition.

Diagnostic methods include, for example, methods to determine whether a subject with an obstructive pulmonary disease (OPD) or cough has chronic obstructive pulmonary disease (COPD) or asthma and methods to assess the efficacy of a treatment for an OPD or cough. The OPD can be, for example, COPD, asthma, acute bronchitis, emphysema, chronic bronchitis, bronchiectasis, cystic fibrosis, and acute asthma; and symptoms include, for example, coughing, shortness of breath, and wheezing. In these methods, the ATP or ATP agonistic analog solution or composition is administered to the subject by intrapulmonary inhalation or intravenous bolus injection. Further details of these methods can be obtained from U.S. Patent Application Publication No. US 2006/0029548, the entire disclosure of which is incorporated herein by reference in its entirety.

Another diagnostic use for ATP reagent solutions or compositions is in the detection of coronary artery disease and myocardial ischemia. The test can be performed with a solution or composition containing ATP (or an agonistic analog thereof). The test can be performed, for example, by intravenous infusion of any of the solutions (see above) or compositions (see below) containing ATP (or an agonistic analog thereof) in combination with thallium-201 scintigraphy (Ferreira et al. (1995) Rev. Port. Cardiol. 14(3):215-224, 188 (abstract), the disclosure of which is incorporated herein by reference in its entirety). Other such methods employing solutions or compositions disclosed herein include those described in: Coma-Canella et al. (2003) Rev. Esp. Cardiol. 56(4):354-360; Iwado et al. (2002) Eur. J. Nucl. Med. 29:984-990; Kubo et al. (2004) J. Nucl. Med. 45:730-738; Kurata et al. (2005) Circ. J. 69:550-557; Miyazono et al. (1998) Am. J. Cardiol. 82:290-294; Miyagawa et al. (1995) JACC 26:1196-1201; Ohba et al. (2008) Journal of Cardiology 52:30-38; Ohtaki et al. (2008) Ann. Nucl. Med. 22:185-190; Takeuchi et al. (2002) Circ. J. 66:167-172; Teragawa et al. (1999) J. Nucl. Cardiol. 6:324-331; Watanabe et al. (1997) J. Nucl. Med. 38-577-581; Yamada et al. (1994) Am. J. Cardiol. 74:940-941; and Yoneyama et al. (2005) Ann. Nucl. Med. 19(2):83-89, the disclosures of all of which are incorporated herein by reference in their entirety.

Methods of treatment include administering to a subject with an OPD or cough (by any of the routes recited herein, but preferably by pulmonary inhalation or intravenous injection (e.g., intravenous bolus injection) or infusion) any one of the solutions or compositions recited in this disclosure containing an antagonistic ATP analog. The antagonistic ATP analog is preferably a P2X receptor antagonist (e.g., a P2X₃ or a P2X_{2/3} receptor antagonist). The OPD can be any of those disclosed herein. Further details of these methods can be obtained from U.S. Patent Application Publication No. US2006/0029548, the entire disclosure of which is incorporated herein by reference in its entirety.

In addition, solutions and compositions of this disclosure containing an antagonistic ATP analog (e.g., oATP) can be used (by administration to appropriate subjects) to treat neuronal damage. The antagonistic ATP analog is preferably a P2X receptor antagonist (e.g., a P2X₄ or a P2X₇ receptor antagonist). The damage can be due to increased extracellular (e.g., intraocular) pressure and the involved neurons can be CNS or PNS neurons. Of particular interest are retinal neurons (e.g., retinal ganglion cells). These methods are discussed in greater detail in U.S. patent application Ser. No. 11/916,237, the disclosure of which is incorporated herein by reference in its entirety.

Furthermore, a solution or composition containing ATP, or an agonistic analog thereof, can be administered to a female subject immediately before, during, or immediately after sexual intercourse in order to contact spermatozoa in her reproductive tract with the ATP, or agonistic analog thereof, and thereby enhance the motility of the spermatozoa. The solution or composition can be administered directly to the female reproductive tract (e.g., intravaginally). For further details of such methods, see published International Patent Application No. WO 2008/106527, the disclosure of which is incorporated herein by reference in its entirety.

Moreover, ATP (or an agonistic analog thereof) in any or the solutions of compositions of this disclosure can be administered to relevant subjects having pain (e.g., burn-related, chemically induced pain, surgery-related pain, infection-related (e.g., postherpetic) pain), organ failure, a condition requiring reduction in blood pressure, pulmonary hypertension, tachycardia, ischemic coronary artery disease, cystic fibrosis, cancer, and cancer-related cachexia. Cancers can be any of those listed herein. Further details on these methods can be found in Agteresch et al. ((1999) Drugs 58(2): 211-232), references cited therein, and Hayashida et al. (2005) J. Anesth. 19:31-35, the disclosures of all of which are incorporated herein by reference in their entirety.

### Ex Vivo Methods

Ex vivo methods involve obtaining a plurality of cells from a subject, contacting them in vitro with an ATP reagent (see above), using most commonly ATP or an agonistic ATP analog solution or composition, and then returning (e.g., by implantation, or infusion or injection by any of the routes recited below) them to the subject. Alternatively, after the contacting, the cells can be administered to a different subject. Moreover, prior to returning them to the original subject or administering them to a different subject, the cells can be treated to prevent division (e.g., by exposing them to ionizing radiation (e.g., x- or γ-irradiation) or treating them with an anti-mitotic drug such as mitomycin C).

One application of such an ex vivo method is in depletion of cancer cells (e.g., leukemia cells) from a source of stem cells (e.g., bone marrow, blood, or umbilical cord). After removal from a subject of a stem cell source that is known, or is suspected, to be contaminated with cancer cells, and prior to administering the stem cell-containing cell population back to the subject (or to a different subject), it can be contacted one or more times with ATP or an agonistic ATP analog using the above-described in vitro methodology so as to kill the cancer cells in the stem cell population. See, for example, Hatta et al. (1993) Intern. Med. 32(10): 768-772; and Hatta et al. (1994) Leuk. Res. 18(8): 637-641, the disclosures of which are incorporated herein by reference in their entirety.

### Pharmaceutical Compositions and Methods of Treatment

Any of the ATP reagent solutions described herein can be, or can be incorporated into, pharmaceutical compositions. Where the solutions are incorporated into pharmaceutical compositions, the incorporation can occur immediately after the solution is made or after storage of the solution (e.g., under the conditions described above). Such compositions typically include at least one pharmaceutically acceptable carrier. As used herein the language “pharmaceutically acceptable carrier” includes solvents, dispersion media, antibacterial and antifungal agents, isotonic and absorption delaying agents, and the like, compatible with pharmaceutical administration. A compound as described herein can be formulated for administration to a subject as a pharmaceutical composition in the form of a syrup, an elixir, a liquid-containing capsule, an aqueous solution, a cream, an ointment, a lotion, a gel, or an emulsion. Supplementary active compounds can also be incorporated into the compositions.

A pharmaceutical solution or composition can be formulated to be compatible with its intended route of administration. Examples of routes of administration include parenteral, e.g., intravenous, intraarterial, intramuscular, intracardiac, intraosseus, intradermal, intrathecal, intradermal, intraperitoneal, intravesical, intravaginal, subcutaneous, inhalational (e.g., by pulmonary inhalation), transdermal, or transmucosal. Intravenous, intraarterial, intramuscular, intracardiac, intraosseus, intradermal, intrathecal, intradermal, intraperitoneal, and subcutaneous administrations are most commonly by injection or infusion. Enteral routes of administration include oral, rectal, and gastric or duodenal (e.g., via a gastric or duodenal tube). Topical routes include epicutaneous and other routes where the composition is applied at the site required. Compositions (e.g., those used for injection or infusion by any of the above parenteral routes) can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. A parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). For injection, the composition must be sterile and should be fluid to the extent that easy syringability exists. It should be stable under the conditions of manufacture and storage and must be preserved against the contamination by microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of infections by microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable pharmaceutical solutions and compositions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation can include vacuum drying or freeze-drying which yields a powder of the active ingredient plus any additional desired ingredient from a previously sterile-filtered solution thereof.

Oral pharmaceutical compositions generally include an inert diluent or an edible carrier. For the purpose of oral therapeutic administration, the solutions can be incorporated with excipients and used in the form of capsules, e.g., gelatin capsules. Oral compositions can also be prepared using a fluid carrier for use as a mouthwash. Pharmaceutically compatible binding agents, and/or adjuvant materials can be included as part of the composition. The capsules and the like can contain any of the following ingredients, or compounds of a similar nature: gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Aqueous pharmaceutical compositions suitable for oral use can be prepared by adding to an appropriate ATP reagent solution suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

For administration by inhalation, the appropriate pharmaceutical ATP reagent solutions or compositions are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the ATP reagent solutions are formulated into ointments, salves, gels, or creams as generally known in the art.

The ATP reagent pharmaceutical compositions can also be prepared in the form of suppositories (e.g., with conventional suppository bases such as cocoa butter and other glycerides) or retention enemas for rectal delivery.

In one embodiment, the compositions are prepared with carriers that will protect the ATP reagent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, the disclosure of which is incorporated herein by reference in its entirety.

It can be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of ATP reagent calculated to produce the desired therapeutic effect in association with the required pharmaceutical carrier. Dosage units can also be accompanied by instructions for use.

The dose administered to a subject, in the context of the present disclosure, should be sufficient to affect a beneficial therapeutic response in the subject over time. The dose will be determined by the efficacy of the ATP reagent employed and the condition of the subject, as well as the body weight or surface area of the subject to be treated. The size of the dose also will be determined by the existence, nature, and extent of any adverse side effects that accompany the administration of a particular compound in a particular subject. In determining the effective amount of the compound to be administered in the treatment or prophylaxis of the disease being treated, the physician can evaluate factors such as the circulating plasma levels of the compound, compound toxicities, and/or the progression of the disease, etc. In general, the dose equivalent of an ATP reagent is from about 1 μg/kg to about 100 mg/kg for a typical subject. Many different administration methods are known to those of skill in the art (see above).

For administration, ATP reagents of the present disclosure can be administered at a rate determined by factors that can include, but are not limited to, the pharmacokinetic profile of the ATP reagent, contraindicated drugs, and the side effects of the ATP reagent at various concentrations, as applied to the mass and overall health of the subject. Administration can be accomplished via single or divided doses.

Toxicity and therapeutic efficacy of such compounds can be determined by known pharmaceutical procedures in cell cultures or experimental animals (animal models of cancer, inflammatory disorders, ischemic disorders, or neurodegenerative disorders). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. ATP reagents that exhibit high therapeutic indices are preferred. While ATP reagents that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of ATP reagents lies generally within a range of circulating concentrations that include the ED₅₀ with little or no toxicity. The dosage may vary within this range depending upon the dosage form employed and the route of administration utilized. For a compound used as described herein, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As used herein, a compound that is “therapeutic” is a compound that causes a complete abolishment of the symptoms of a disease or a decrease in the severity of at least one symptom of the disease. “Prevention” means that symptoms of the disease (e.g., cancer) are essentially absent. As used herein, “prophylaxis” means complete prevention of the symptoms of a disease, a delay in onset of the symptoms of a disease, or a lessening in the severity of subsequently developed disease symptoms.

The term “therapeutically effective amount” or “therapeutically effective dose” is intended to mean that amount of a compound (e.g., ATP) that will elicit the desired biological or medical response. For example, a “therapeutically effective amount” of a compound (e.g., ATP) can be one that ameliorates one or more symptoms of a subject's pathological condition such as any of those described herein. As defined herein, a therapeutically effective amount of a compound (e.g., ATP) (i.e., an effective dosage) includes milligram or microgram amounts of the compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a compound depend upon the potency of the compound with respect to an activity of interest, e.g., enhancement of spermatozoon motility or inhibition of cancer cell division. When one or more of ATP reagents are to be administered to an animal (e.g., a human) to treat a condition, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific ATP reagent employed, the age, body weight, general health, gender, and diet of the subject, the time of administration, the route of administration, the rate of excretion, any drug combination, and the degree of expression or activity to be modulated. One in the art will also appreciate that certain additional factors may influence the dosage and timing required to effectively treat a subject, including but not limited to the severity of the disease or disorder, previous treatments, and other diseases present. Moreover, treatment of a subject with a therapeutically effective amount of an ATP reagent (e.g., ATP) can include a single treatment or can include a series of treatments.

As defined herein, a “prophylatically effective amount” of a compound (e.g., ATP) is an amount of the compound that is capable of producing total prevention of, a decrease in the severity of, or a delay in the onset of symptoms of a condition of interest. A prophylatically effective amount of a reagent (i.e., an effective dosage) includes milligram, microgram, nanogram, or picogram amounts of the reagent per kilogram of subject or sample weight (e.g., about 1 nanogram per kilogram to about 500 micrograms per kilogram, about 1 microgram per kilogram to about 500 milligrams per kilogram, about 100 micrograms per kilogram to about 5 milligrams per kilogram, or about 1 microgram per kilogram to about 50 micrograms per kilogram).

The pharmaceutical compositions and solutions of the disclosure can be included as components of kits in a container, pack, or dispenser together with instructions for administration. The disclosure also provides kits containing any of the above described solution components and instructions for making the solution. Such components, which can be, e.g., one or more ATP reagents and/or glycine and/or Na₂HPO₄, can be provided in the kits in powder or crystalline form or dissolved in a solvent, preferably an aqueous solvent.

The following Example serves to illustrate, not limit, the invention.

### EXAMPLE

An aqueous solution containing the following components was made:

10 mg/ml of adenosine 5′-triphosphate disodium salt (anhydrous) (18.15 mM)

1 mg/ml of glycine (13.32 mM)

36 mg/ml of Na₂HPO₄.7H₂O (134.3 mM)

Sodium hydroxide (sufficient to adjust pH to 9.2-9.3)

Water sufficient to result in the above concentrations of adenosine 5′-triphosphate disodium salt (anhydrous), glycine, and 36 mg/ml of Na₂HPO₄.7H₂O.

The concentrations of ATP and ADP in the solution were measured immediately after making the solution (0 time sample) and in aliquots stored in a refrigerator (at an ambient temperature in the range of about 4° C. to 8° C. for 1 month, 3 months, 6 months, 12 months, 24 months, 36 months, and 72 months. The presence of other potential ATP degradation (hydrolysis) products (AMP and adenosine) was also tested for and significant levels of neither were detectable at any of the time points. The concentrations of the compounds were measured by high pressure liquid chromatography (HPLC).

Below (in Table 2) are shown the relative amounts of ADP in the solution aliquots at the various time points. The data shown are concentrations of ADP as a percentage of the concentrations of ATP in the solution aliquots. The pH of the 72 month time point sample was 8.8.

TABLE 2
Relative amounts of ADP in solutions of ATP stored for various
lengths of time at a temperature of about 4° C. to about 8° C.

| Time of storage | Relative amount of ADP |
|---|---|
| (in months) | ([ADP]/[ATP] × 100) (%) |
| 0 | 0.35 |
| 1 | 0.42 |
| 3 | 0.48 |
| 6 | 0.57 |
| 12 | 0.72 |
| 24 | 1.15 |
| 36 | 1.4 |
| 72 | 2.9 |

A number of embodiments of the invention have been described. Nevertheless, it will be understood that various modifications may be made without departing from the spirit and scope of the invention. Accordingly, other embodiments are within the scope of the following claims.

What is claimed is:

1. An aqueous solution comprising a mixture selected from the group consisting of:
(a) an aqueous solvent,
an adenosine 5-triphosphate (ATP) reagent, and
glycine,
wherein the solution has a pH of between about 8.7 and about 9.5;
(b) an aqueous solvent, and
an ATP reagent,
wherein the solution has a pH of about 8.7 to about 9.5 and wherein the solution is formulated for administration to a subject or for contacting a mammalian cell with the ATP or the analog thereof; and
(c) an aqueous solvent, and
ATP,
wherein the solution has a pH of between about 8.7 and about 9.5,
such that the solution, at the end of a period of time at about 4° C. to about 8° C., contains an amount of ADP that is not more than about 5.0% of the amount of ATP in the solution, the period of time being up to six years after the solution was made.

2. The solution of claim 1, wherein:
(i) not more than about 5% of the ATP is not more than about 4.0% of ATP that the solution contained when the solution was made;
(ii) not more than about 5% of the ATP is not more than about 3.0% of ATP that the solution contained when the solution was made; or
(iii) not more than about 5% of the ATP is not more than about 2.5% of ATP that the solution contained when the solution was made.

3. The aqueous solution claim 1, wherein the ATP reagent is: ATP or a pharmaceutically acceptable salt thereof; or an ATP analog or a pharmaceutically acceptable salt thereof.

4. The solution of claim 1, wherein (b) and (c) further comprise glycine.

5. The solution of claim 1, wherein the solution has a pH of between about 8.7 and about 9.4 or between about 8.8 and about 9.3.

6. The solution of claim 1, further comprising one or both of a biocompatible buffer and a stabilizer.

7. The solution of claim 6, wherein the biocompatible buffer is a phosphate buffer.

8. The solution of claim 7, wherein the phosphate buffer comprises Na₂HPO₄.

9. The solution of claim 6, wherein the biocompatible buffer is a bicarbonate buffer, an acetate buffer, a citrate buffer, or a glutamate buffer.

10. The solution of claim 6, wherein the solution comprises 2-(N-morpholino)ethanesulfonic acid (MES), tris(hydroxymethyl)aminomethane (Tris), (N-[2-hydroxyethyl]piperazine-N′-[2-ethanesulfonic acid] (HEPES), 3{[tris(hydroxymethyl)methyl]amino}propanesulfonic acid (TAPS), N,N-bis(2-hydroxymethyl)glycine (Bicine), N-tris(hydroxymethyl)methylglycine (Tricine), 2-{[tris(hydroxymethyl)methyl]amino}ethanesulfonic acid (TES), 3-(N-morpholino)propanesulfonic acid (MOPS), piperazine-N,N′-bis(2-ethanesulfonic acid) (PIPES), dimethylarsinic acid (Cacodylate), or 2-(N-morpholino)ethanesulfonic acid (MES).

11. The solution of claim 6, wherein the stabilizer is:
a chelating agent selected from the group consisting of ethylenediaminetetraacetic acid (EDTA) and ethylene glycol tetraacetic acid (EGTA);
a sugar alcohol selected from the group consisting of sorbitol, mannitol, adonitol, erythritol, xylitol, lactitol, isomalt, maltitol, or a cyclitol;
glycerol;
methionine; and
creatinine.

12. The solution of claim 3, wherein the ATP analog is selected from the group consisting of: α,β-methylene-ATP (α,βmATP); β,γ-methylene-ATP (β,γmATP); 2-thio-ATP (2-SH-ATP); 2-methylthio-ATP (2-MeS-ATP); 2′,3′-O-2,4,6,-trinitrophenyl-ATP (TNP-ATP); 2′,3′-O-(4-benzoyl)-ATP (BzATP); an N-alkyl-2 ATP; adenosine 5′-(, -imido)triphosphate (AMP-PNP); ATP-MgCl2; and oxidized ATP (oATP).

13. The solution of claim 1, wherein concentration of the ATP reagent in the solution is: about 18.15 mM; about 36.30 mM; or about 13.32 mM.

14. The solution of claim 1, wherein the solution comprises:
18.15 mM ATP; and
Na₂HPO₄.

15. The solution of claim 1, wherein the solution is formulated for: parenteral administration to a subject; administration to a subject by inhalation; intravenous administration to a subject; enteral administration to a subject; oral administration to a subject; topical administration to a subject; or transdermal administration to a subject.

16. A method of making an aqueous solution, the method comprising:
mixing together
water,
glycine,
and an ATP reagent to create a mixture; and
adjusting the pH of the mixture to between about 8.7 to about 9.5 to create the solution.

17. The method of claim 16, further comprising mixing into the mixture a biocompatible buffer.

18. The method of claim 16, further comprising mixing into the mixture a stabilizer.

19. The method of claim 16, further comprising storing the solution at a temperature of between about 4° C. and about 8° C. for a period of time of up to six years.

20. The method of claim 19, wherein the ATP reagent is ATP and the solution, at the end of the period of time, contains an amount of ADP that is not more than about 5.0% of the amount of ATP in the solution.

21. An in vitro method of delivering an ATP reagent to a mammalian cell, the method comprising incubating the cell with a medium comprising the solution of claim 1 in vitro.

22. The method of claim 21, wherein the mammalian cell is spermatozoon.

23. An in vivo method of contacting a mammalian cell in a mammalian subject with an ATP reagent, the method comprising administering a composition comprising the solution of claim 1 to the subject.

24. The method of claim 23, wherein the subject is a human subject.

25. The method of claim 23, wherein the method is a therapeutic method, a prophylactic method, or a diagnostic method.

26. The method of claim 23, wherein the subject has, is suspected of having, or is at risk of developing a condition selected from the group consisting of an obstructive pulmonary disease (OPD), asthenozoospermia, pain, tissue injury, nerve damage, organ failure, a condition requiring reduction in blood pressure, pulmonary hypertension, tachycardia, myocardial ischemia, coronary artery disease, cystic fibrosis, cancer, and cancer-related cachexia.

27. The method of claim 23, wherein the cell is a neuron, a spermatozoon, a vascular smooth muscle cell, a vascular endothelial cell, or a cancer cell.

28. The method of claim 27, wherein the neuron is retinal neuron, a cortical neuron, a hippocampal neuron, a basal ganglion neuron, a spinal cord neuron, a pulmonary vagal C fiber, or a pulmonary vagal A fiber.

29. The method of claim 26, wherein the OPD is selected from the group consisting of chronic obstructive pulmonary disease (COPD), chronic asthma, acute bronchitis, emphysema, chronic bronchitis, bronchiectasis, cystic fibrosis, cough, and acute asthma.

30. The method of claim 23, wherein the method is a method: (a) to determine whether the subject has COPD or asthma; (b) for assessing the efficacy of a treatment for an OPD.

31. The method of claim 22, further comprising testing the motility of the spermatozoon.

32. A kit comprising:
(a) the solution of claim 1 and instructions for administering the solution to a subject; or
(b) an ATP reagent, glycine, and instructions for making the solution of claim 1.

33. The kit of claim 32, wherein (b) further comprises Na₂HPO4.
